(19) United States
(12) Patent Application Publication
(10) Pub. No.: US 2006/0293632 A1
(43) Pub. Date: Dec. 28, 2006

(54) Absorbent article featuring a non-abrasive temperature change member

(75) Inventors: Andrew M. Long, Appleton, WI (US); Davis Dang Nhan, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/512,801

(22) Filed: Aug. 29, 2006

Related U.S. Application Data
(63) Continuation-in-part of application No. 11/299,433, filed on Dec. 12, 2005.
Application No. 11/299,433 is a continuation-in-part of application No. 11/143,359, filed on Jun. 1, 2005.
Application No. 11/143,359 is a continuation-in-part of application No. 11/025,188, filed on Dec. 29, 2004.

(51) Int. Cl. A61F 13/15

(52) U.S. Cl. 604/361

(57) ABSTRACT

Disclosed is an absorbent article including a temperature change member. In addition to the temperature change member, the article may include an outercover, with a liner and/or an absorbent body disposed on the outercover. The temperature change member has a geometric mean of the mean deviation of coefficient of friction that is less than about 0.0275 when tested at the surface intended to face a wearer.

This application is a continuation-in-part of U.S. patent application Ser. No. 11/299,433 filed Dec. 15, 2005, and entitled “Absorbent Article Featuring a Non-Abrasive Temperature Change Member,” which is a continuation-in-part of U.S. patent application Ser. No. 11/143,359 filed Jun. 1, 2005, and entitled “Absorbent Article Featuring a Temperature Change Member,” which is a continuation-in-part of U.S. patent application Ser. No. 11/025,188 filed Dec. 29, 2004, and entitled “Absorbent Article Featuring a Temperature Change Member.” The entirety of application Ser. Nos. 11/025,188, 11/299,433 and 11/143,359 is hereby incorporated by reference.

### BACKGROUND OF THE INVENTION

The present invention relates to absorbent articles that include a temperature change member. More specifically, the invention relates to an absorbent article such as training pants that provides the wearer with a noticeable temperature change sensation upon urination.

Absorbent articles, such as children's training pants, have been designed with temperature change members to provide a temperature change sensation upon urination in an attempt to enhance a child's recognition of when urination occurs. As can be appreciated, such recognition can be an important step in the toilet training process. The temperature change sensation provided by the temperature change member can often be the result of temperature change material that is disposed with the temperature change member.

Unfortunately, in certain circumstances, such temperature change members may not be completely satisfactory. For example, the temperature change material included with the temperature change member can, in certain instances, be abrasive to the wearer. This abrasiveness can be particularly notable where the temperature change member is maintained close to the wearer in use, which is generally a desirable configuration to maximize the temperature change sensation experienced by the wearer. Moreover, the temperature change member may provide a rapid temperature change sensation, but it may not last as long as desired to assist with the toilet training process. Thus, there is a need for an absorbent article with a temperature change member that is effective and yet provides a pleasing surface to the wearer.

### SUMMARY OF THE INVENTION

Provided is an absorbent article defining a longitudinal direction, a lateral direction perpendicular to the longitudinal direction, and a z-direction perpendicular to a plane, defined by the lateral and longitudinal directions. The absorbent article includes an outercover, an absorbent body disposed on the outercover, and a liner disposed on the absorbent body. A temperature change member is disposed between the liner and the outercover. The temperature change member has temperature change material disposed in a nonuniform distribution by weight in the z-direction of the temperature change member. The temperature change member defines a member inner surface and a member outer surface opposite the member inner surface, and has a GMD of less than about 0.0275 at the member inner surface, as determined by the friction test described herein.

In another aspect of the invention, an absorbent article includes a liquid impermeable outercover, an absorbent body disposed on the outercover, and a temperature change member disposed within the absorbent body. The temperature change member includes temperature change material wherein at least 50 percent of the temperature change material defines a particle size of at least 500 microns by weight. The absorbent article has a GMD, as determined by the friction test described herein, of less than 0.0270 when tested at the member inner surface.

In yet another aspect of the invention, an absorbent article defines a longitudinal direction, a lateral direction perpendicular to the longitudinal direction, and a z-direction perpendicular to a plane defined by the lateral direction and the longitudinal direction. The absorbent article includes a liquid impermeable outercover having a garment-facing surface and a body-facing surface and a temperature change member disposed on the outercover body-facing surface. The temperature change member defines a member inner surface and a member outer surface opposite the member inner surface, and has a temperature change material disposed in a nonuniform distribution by weight in the z-direction of the temperature change member. The temperature change member has a GMD of less than about 0.0275 at the member inner surface, as determined by the friction test described herein.

The above-mentioned and other aspects of the present invention will become more apparent, and the invention itself will be better understood by reference to the drawings and the following description of the drawings

### BRIEF DESCRIPTION OF THE DRAWINGS

FIG. 1 representatively illustrates a side view of a pair of training pants with a mechanical fastening system of the pants shown fastened on one side of the training pants and unfastened on the other side of the training pants;

FIG. 2 representatively illustrates a plan view of the training pants of FIG. 1 in an unfastened, stretched and laid flat condition, and showing the surface of the training pants that faces away from the wearer;

FIG. 3 representatively illustrates a plan view similar to FIG. 2, but showing the surface of the training pants that faces the wearer when worn, and with portions cut away to show underlying features;

FIG. 4 representatively illustrates a section view of a particular aspect of the temperature change member of the present invention;

FIG. 5 representatively illustrates a section view of another aspect of the temperature change member of the present invention;

FIG. 6 representatively illustrates a section view of yet another aspect of the temperature change member of the present invention;

FIG. 7 representatively illustrates a section view of still yet another aspect of the temperature change member of the present invention;

FIG. 8 representatively illustrates a section view of another aspect of the training pants with elements removed for clarity; and

FIG. 9 representatively illustrates a section view of another aspect of the training pants with elements removed for clarity.

Corresponding reference characters indicate corresponding parts throughout the drawings.

Definitions

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 50 percent (to 150 percent) of its relaxed length and will recover, upon release of the applied force, at least 40 percent of its elongation.

“Extensible” refers to a material or composite which is capable of extension or deformation without breaking, but does not substantially recover its original size and shape after removal of a force causing the extension or deformation. Suitably, an extensible material or composite can be elongated by at least 50 percent (to 150 percent) of its relaxed length.

“Fiber” refers to a continuous or discontinuous member having a high ratio of length to diameter or width. Thus, a fiber may be a filament, a thread, a strand, a yarn, or any other member or combination of these members.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Join” and its derivatives refer to the connecting, adhering, bonding, attaching, sewing together, or the like, of two elements. Two elements will be considered to be joined together when they are integral with or joined directly to one another or indirectly to one another, such as when each is directly joined to intermediate elements. “Join” and its derivatives include permanent, releasable, or refastenable joinder. In addition, the joining can be completed either during the manufacturing process or by the end user.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Such a process is disclosed, for example, in U.S. Pat. No. 3,849,241 to Butin et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Particle” and its derivatives refer to discrete portions of a material and can include crystals, agglomerates of particles and the like.

“Stretchable” means that a material can be stretched, without breaking, by at least 50 percent (to 150 percent of its initial (unstretched) length) in at least one direction. Elastic materials and extensible materials are each stretchable materials.

“Superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms may be defined with additional language in the remaining portions of the specification.

### DETAILED DESCRIPTION

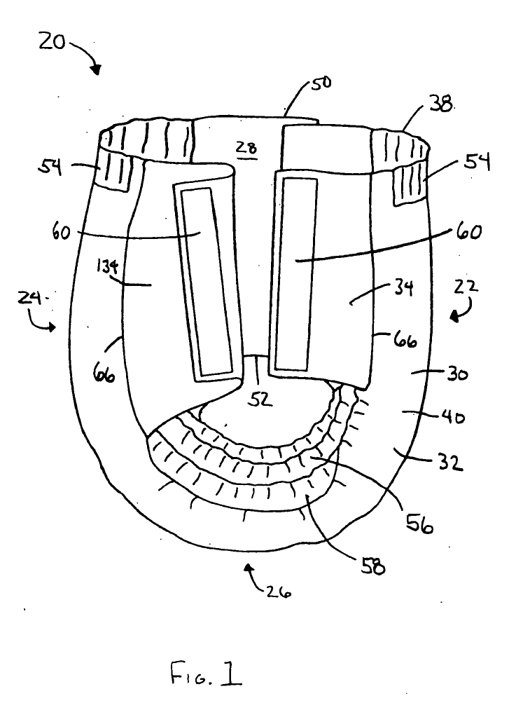

Referring now to the drawings and in particular to FIG. 1, an absorbent article of the present invention is representatively illustrated in the form of children's toilet training pants and is indicated in its entirety by the reference numeral 20. The pants 20 include a temperature change member 70 that is adapted to create a distinct temperature change sensation to the wearer upon urination, which can enhance a wearer's ability to recognize when urination is occurring.

The pants 20 may or may not be disposable, which refers to articles that are intended to be discarded after a limited period of use instead of being laundered or otherwise conditioned for reuse. It should also be understood that the present invention can be suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson, et al. which are incorporated herein by reference to the extent that they are consistent (i.e., not in conflict) herewith. In addition, absorbent articles including a temperature change member are described in U.S. Pat. No. 5,681,298 to Brunner et al., U.S. patent application Ser. No. 11/143,359 filed Jun. 1, 2005 in the name of Jackson, et al., U.S. patent application Ser. No. 11/246,414 filed Oct. 7, 2005, in the name of Olson, and U.S. patent application Ser. No. 11/245,870 filed Oct. 7, 2005, in the name of Olson, the disclosures of which are incorporated herein by reference to the extent that they are consistent (i.e., not in conflict) herewith.

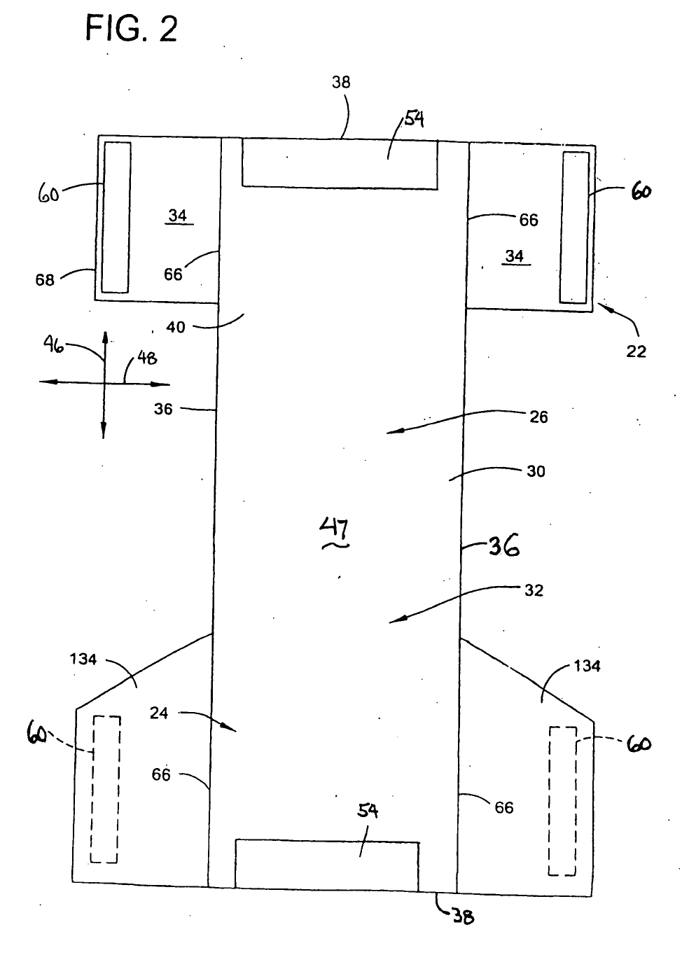

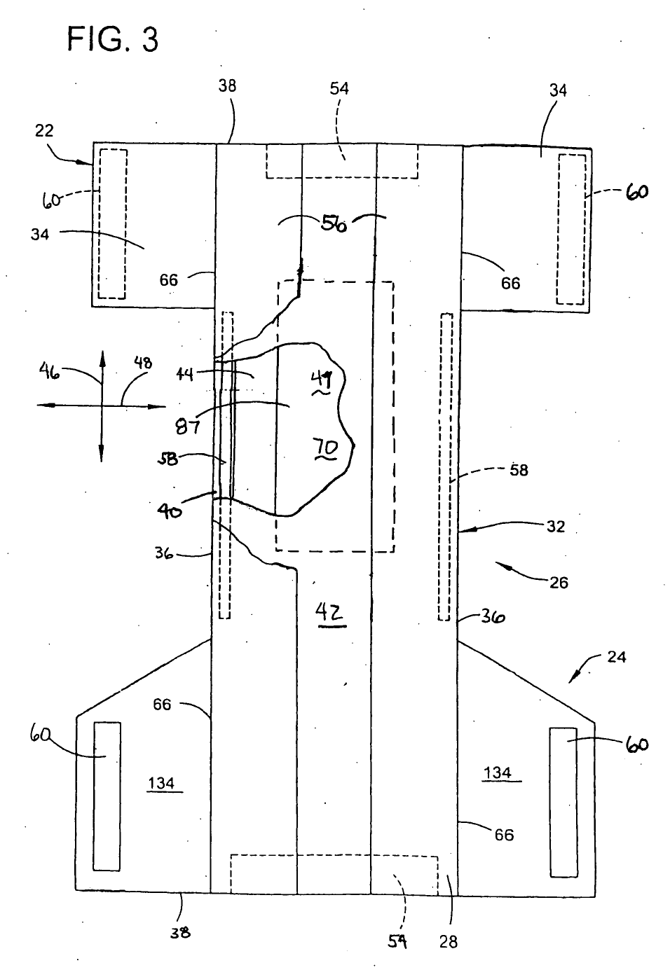

The training pants 20 are illustrated in FIG. 1 in a partially fastened condition. The pants 20 define a longitudinal direction 46 and a lateral direction 48 perpendicular to the longitudinal direction as shown in FIGS. 2 and 3. In addition, the pants 20 define a z-direction 49 that is perpendicular to a plane 47 defined by the lateral direction 48 and the longitudinal direction 46. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 includes those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The pants 20 also define an inner surface 28 adapted in use to be disposed toward the wearer, and an outer surface 30 opposite the inner surface. With additional reference to FIGS. 2 and 3, the pair of training pants 20 has a pair of longitudinally extending side edges 36, and a pair of laterally extending opposing waist edges 38 (broadly, end edges).

The illustrated pants 20 can include an absorbent assembly, generally indicated at 32. For example, in the aspect of FIGS. 1-3, the training pants 20 can include a generally rectangular central absorbent assembly 32 and side panels 34, 134 formed separately from and secured to the central absorbent assembly. The side panels 34, 134 can be bonded along seams 66 to the absorbent assembly 32 in the respective front and back waist regions 22 and 24 of the pants 20. More particularly, the front side panels 34 can be permanently bonded to and extend laterally outward from the absorbent assembly 32 at the front waist region 22, and the back side panels 134 can be permanently bonded to and extend laterally from the absorbent assembly 32 at the back waist region 24. The side panels 34 and 134 may be bonded to the absorbent assembly 32 using attachment means known to those skilled in the art such as adhesive, thermal, pressure or ultrasonic bonding.

The front and back side panels 34 and 134, upon wearing of the pants 20, thus include the portions of the training pants 20 that are positioned on the hips of the wearer. The front and back side panels 34 and 134 can be permanently bonded together to form the three-dimensional configuration of the pants 20, or be releasably connected with one another such as by a fastening system 60.

Suitable elastic materials, as well as processes for incorporating elastic side panels into training pants, are described in the following U.S. Patents: 4,940,464 issued Jul. 10, 1990 to Van Gompel et al.; 5,224,405 issued Jul. 6, 1993 to Pohjola; 5,104,116 issued Apr. 14, 1992 to Pohjola; and 5,046,272 issued Sep. 10, 1991 to Vogt et al.; all of which are incorporated herein by reference in their entirety to the extent they are consistent (i.e., not in conflict) herewith. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in their entirety to the extent they are consistent (i.e., not in conflict) herewith. As is known in the art, the side panels 34, 134 may include elastic material or stretchable but inelastic materials.

The absorbent assembly 32 is illustrated in FIGS. 2-3 as having a rectangular shape. However, it is contemplated that the absorbent assembly 32 may have other shapes (e.g., hourglass, T-shaped, I-shaped, and the like) without departing from the scope of this invention. It is also understood that the side panels 34, 134 may alternatively be formed integrally with the absorbent assembly 32 without departing from the scope of this invention. In such a configuration, the side panels 34 and 134 and the absorbent assembly 32 would include at least some common materials, such as the bodyside liner 42, outercover 40, other materials and/or combinations thereof.

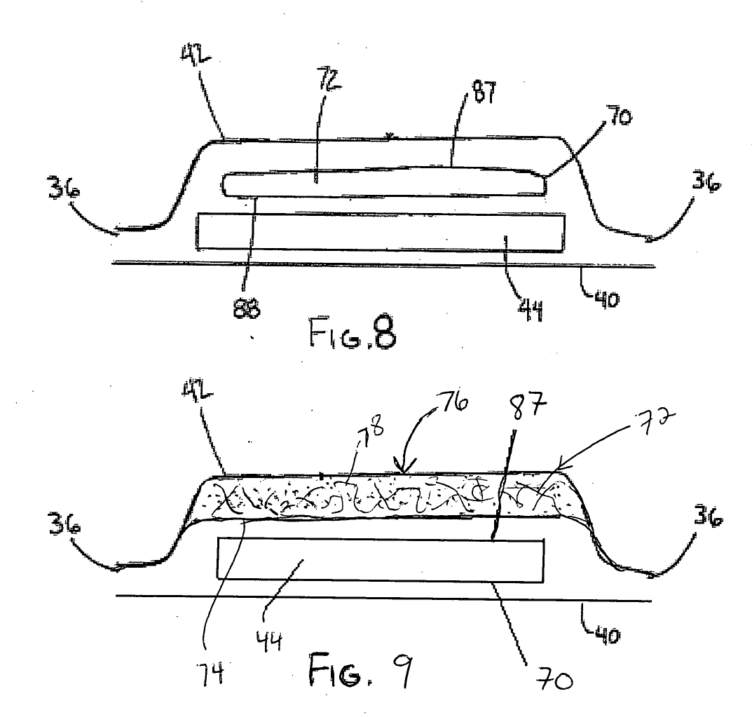

The absorbent assembly 32 includes an outercover 40 and a bodyside liner 42 (FIGS. 3, 8) in a superposed relation therewith. The liner 42 can be suitably joined to the outercover 40 along at least a portion of the absorbent assembly 32. The liner 42 can be suitably adapted, i.e., positioned relative to the other components of the pants 20, to contact the wearer's skin during wear of the pants. The absorbent assembly 32 also includes an absorbent body 44 for absorbing liquid body exudates disposed on the outercover 40 (FIGS. 3, 8). In a particular aspect, the absorbent body 44 can be sandwiched between the outercover 40 and the bodyside liner 42. The bodyside liner 42 and the outercover 40 can be attached to each other by adhesive, ultrasonic bonding, thermal bonding or by other suitable attachment techniques known in the art. Moreover, at least a portion of the absorbent body 44 can optionally be attached to the bodyside liner 42 and/or the outercover 40 utilizing the methods described above.

As mentioned above, the front and back side panels 34 and 134 can be releasably connected with one another such as by the fastening system 60 of the illustrated aspect. With the training pants 20 in the fastened position as partially illustrated in FIG. 1, the front and back waist regions are connected together to define the three-dimensional pants configuration having a waist opening 50 and a pair of leg openings 52. The waist edges 38 of the training pants 20 are configured to encircle the waist of the wearer to define the waist opening 50 (FIG. 1) of the pants.

The fastening system 60 may include any refastenable fasteners suitable for absorbent articles, such as adhesive fasteners, cohesive fasteners, mechanical fasteners, or the like. In one aspect of the invention, the fastening system includes mechanical fastening elements for improved performance. Suitable mechanical fastening elements can be provided by interlocking geometric-shaped materials, such as hooks, loops, bulbs, mushrooms, arrowheads, balls on stems, male and female mating components, buckles, snaps, or the like. For example, fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The pants 20 may further include a pair of containment flaps 56 for inhibiting the lateral flow of body exudates. As illustrated in FIG. 3, the containment flaps 56 can be operatively attached to the pants 20 in any suitable manner as is well known in the art. In particular, suitable constructions and arrangements for the containment flaps 56 are generally well known to those skilled in the art and are described in U.S. Pat. No. 4,704,116 issued Nov. 3, 1987 to Enloe, which is incorporated herein by reference to the extent that it is consistent (i.e., not in conflict) herewith.

To further enhance containment and/or absorption of body exudates, the training pants 20 may include waist elastic members 54 in the front and/or back waist regions 22 and 24 of the pants 20. Likewise, the pants 20 may include leg elastic members 58, as are known to those skilled in the art. The waist elastic members 54 and the leg elastic members 58 can be formed of any suitable elastic material that is well known to those skilled in the art. For example, suitable elastic materials include sheets, strands or ribbons of natural rubber, synthetic rubber, or thermoplastic elastomeric polymers. In one aspect of the invention, the waist elastics and/or the leg elastics may include a plurality of dry-spun coalesced multi-filament elastomeric threads sold under the trade name LYCRA and available from Invista of Wilmington, Del., U.S.A.

The outercover 40 may suitably include a material that is substantially liquid impermeable. The outercover 40 may be provided by a single layer of liquid impermeable material, or more suitably include a multi-layered laminate structure in which at least one of the layers is liquid impermeable. In particular aspects, the outer layer may suitably provide a relatively cloth-like texture to the wearer. A suitable liquid impermeable film for use as a liquid impermeable inner layer, or a single layer liquid impermeable outercover 40 is a 0.025 millimeter (1.0 mil) polyethylene film commercially available from Edison Plastics Company of South Plainfield, N.J. Alternatively, the outercover 40 may include a woven or non-woven fibrous web layer that has been totally or partially constructed or treated to impart the desired levels of liquid impermeability to selected regions that are adjacent or proximate the absorbent body.

The outercover 40 may also be stretchable, and in some aspects it may be elastomeric. For example, such an outercover material can include a 0.3 osy polypropylene spunbond that is necked 60 percent in the lateral direction 40 and creped 60 percent in the longitudinal direction 48, laminated with 3 grams per square meter (gsm) Bostik-Findley H2525A styrene-isoprene-styrene based adhesive to 8 gsm PEBAX 2533 film with 20 percent TiO₂ concentrate. Reference is made to U.S. Pat. No. 5,883,028, issued to Morman et al., U.S. Pat. No. 5,116,662 issued to Morman and U.S. Pat. No. 5,114,781 issued to Morman, all of which are hereby incorporated herein by reference, for additional information regarding suitable outercover materials.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent body 44. A suitable liquid permeable bodyside liner 42 is a nonwoven polyethylene/polypropylene bicomponent web having a basis weight of about 27 gsm; the web may be spunbonded or a bonded carded web. Optionally, the bodyside liner 42 may be treated with a surfactant to increase the wettability of the liner material.

Alternatively, the bodyside liner 42 may also be stretchable, and in some aspects it may be elastomeric. For instance, the liner 42 can be a non-woven, spunbond polypropylene fabric composed of about 2 to 3 denier fibers formed into a web having a basis weight of about 12 gsm which is necked approximately 60 percent. Strands of about 9 gsm KRATON G2760 elastomer material placed eight strands per inch (2.54 cm) can be adhered to the necked spunbond material to impart elasticity to the spunbond fabric. The fabric can be surface treated with an operative amount of surfactant, such as about 0.6 percent AHCOVEL Base N62 surfactant, available from ICI Americas, a business having offices in Wilmington, Del., U.S.A. Other suitable materials may be extensible biaxially stretchable materials, such as a neck stretched/creped spunbond. Reference is made to U.S. Pat. No. 6,552,245, issued Apr. 22, 2003, to Roessler et al., which is incorporated by reference herein to the extent that it is consistent (i.e., not in conflict) herewith.

An absorbent body 44 may be disposed on the outercover 40, for example, between the outercover 40 and the bodyside liner 42. The outercover 40 and the bodyside liner 42 can be joined together by any suitable means such as adhesives, ultrasonic bonds, thermal bonds, or the like. The absorbent body 44 can be in a variety of shapes and configurations as are known in the art, such as rectangular, hourglass shaped, I-shaped, and the like. Further, at least a portion of the absorbent body 44 can optionally be attached to the bodyside liner 42 and/or the outercover 40 utilizing the methods described above.

The absorbent body 44 is suitably compressible, conformable and capable of absorbing and retaining liquid body exudates released by the wearer. For example, the absorbent assembly can include a matrix of absorbent fibers, and more suitably cellulosic fluff, such as wood pulp fluff, and superabsorbent particles. One suitable pulp fluff is identified with the trade designation CR1654, commercially available from Bowater, Inc. of Greenville, S.C., U.S.A. As an alternative to wood pulp fluff, synthetic fibers, polymeric fibers, meltblown fibers, short cut homofil bicomponent synthetic fibers, or other natural fibers may be used. Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as Dow Chemical Company of Midland, Mich., U.S.A., and Stockhausen Inc., Greensboro, N.C., U.S.A.

In one aspect, the absorbent body 44 may be stretchable so as not to inhibit the stretchability of other components to which the absorbent body may be adhered, such as the outercover 40 and/or the bodyside liner 42. For example, the absorbent body may include materials disclosed in U.S. Pat. Nos. 5,964,743, 5,645,542, 6,231,557, 6,362,389, and international patent application WO 03/051254, the disclosure of each of which is incorporated by reference herein.

In some aspects, a surge management layer (not shown) may be included in the pants 20. The surge management layer may be positioned in the pants 20 in a variety of locations as is known in the art. For example, the surge management layer can be proximate the absorbent body 44, for example between the absorbent body 44 and the bodyside liner 42, and attached to one or more components of the pants 20 by methods known in the art, such as by adhesive, ultrasonic or thermal bonding. In addition, the surge management layer may be positioned in the pants 20 relative to the temperature change member 70 in a variety of ways. For instance, the surge management layer may be disposed toward the liner 42 relative to the temperature change member 70, or the surge management layer may be disposed toward the absorbent body 44 relative to the temperature change member 70.

A surge management layer helps to decelerate and diffuse surges or gushes of liquid that may be rapidly introduced into the absorbent body 44. Desirably, the surge management layer can rapidly accept and temporarily hold the liquid prior to releasing the liquid into the storage or retention portions of the absorbent body 44. Examples of suitable surge management layers are described in U.S. Pat. No. 5,486,166 and U.S. Pat. No. 5,490,846, the contents of which are incorporated herein by reference to the extent that they are consistent (i.e., not in conflict) herewith.

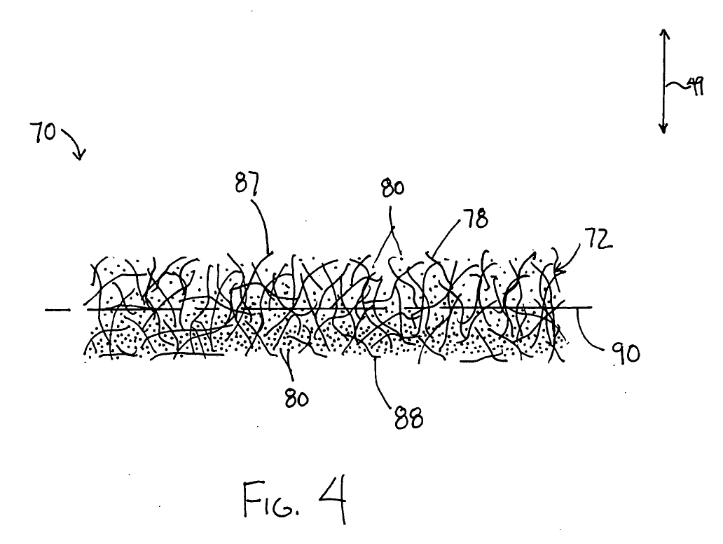

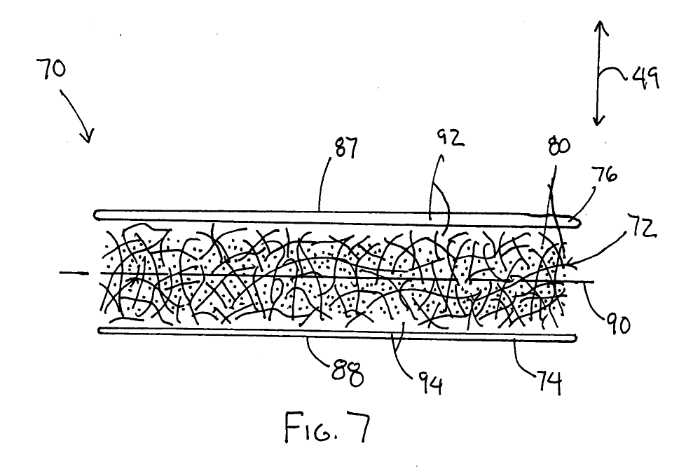

As mentioned above, the various aspects of the absorbent article of the present invention can also include a temperature change member 70 (FIGS. 3-8). The temperature change member 70 can define member inner surface 87 intended to be disposed toward the wearer in use and a member outer surface 88 intended to be disposed away from the wearer in use, opposite the member inner surface (FIGS. 4-8). In addition, as representatively illustrated in FIGS. 4-8, the temperature change member 70 can suitably include a temperature change composite 72.

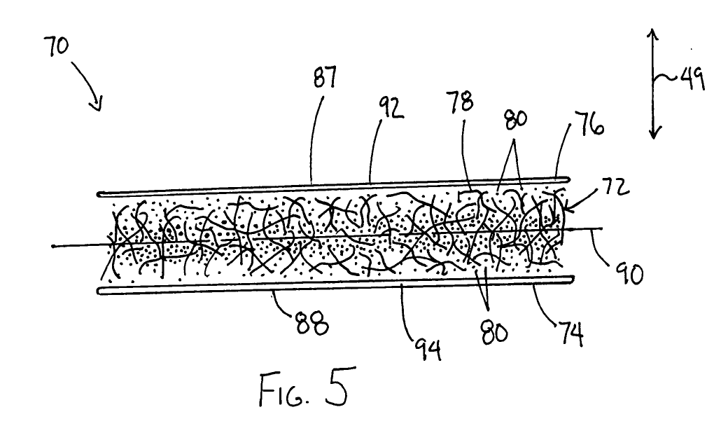

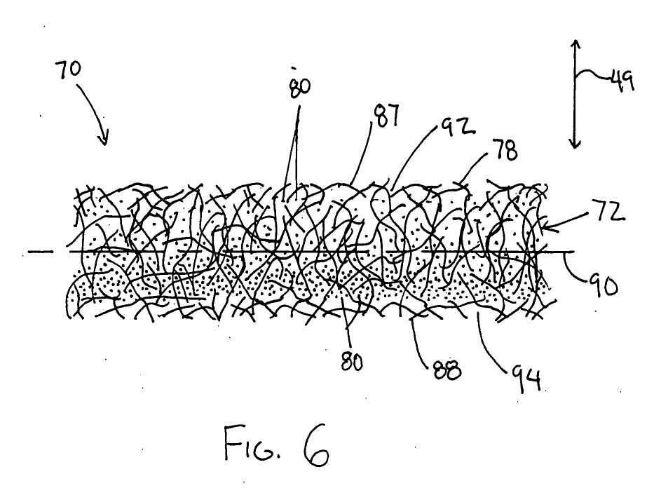

In addition, the temperature change member 70 can include a first carrier layer 74 in a superposed relationship with temperature change composite 72 (FIGS. 5 and 6). The temperature change member 70 can also optionally include a second carrier layer 76 where the first carrier layer 74 and the second carrier layer 76 sandwich the temperature change composite 72 (FIGS. 5-6). As such, in aspects where the temperature change member 70 does not include carrier layers 74 or 76, the composite 72 can provide the surfaces 87 and 88 of the temperature change member 70. Alternatively, when present, the carrier layers 74 and 76 can provide one or both of the temperature change member surfaces 87 and 88.

The temperature change member 70 can suitably include temperature change material 80 (FIGS. 4-7). For example, the temperature change composite 72 can include temperature change material 80 disposed in the temperature change composite 72. As can be appreciated, the temperature change member 70 can define a total amount of temperature change material 80, by weight. For example, in one aspect, the temperature change member 70 can include 1 to 30 grams of material 80, and in particular, 1 to 20 grams of material 80. In yet another alternative, the temperature change member 70 can include 1 to 10 grams of material 80.

The temperature change material 80 can be disposed within the temperature change composite 72 in a variety of configurations. For example, the temperature change material 80 can be distributed in a substantially uniform manner throughout the temperature change composite 72, such that all areas or regions of the temperature change composite 72 has substantially equal amounts of temperature change material 80, measured by weight. Alternatively, the temperature change material 80 can be suitably disposed within the temperature change composite 72 in a nonuniform distribution, measured by weight. In such an aspect, the temperature change material can be strategically located within the temperature change composite 72 to maximize the effectiveness of the temperature change member 70 in use and to present a more pleasing, smooth surface toward the wearer.

Thus, in one aspect, the temperature change material 80 can be disposed within the temperature change composite 72 in a nonuniform distribution by weight through the temperature change composite 72 in the z-direction 49. In such an arrangement, temperature change material 80 can be disposed within the temperature change composite 72 in greater or lesser amounts (measured by weight) in some regions of the temperature change member 70 than in other regions of the temperature change member 70. In particular, in at least one cross-section of the temperature change member 70 having some component in the z-direction 49 (i.e., the cross-section is not taken exclusively in the plane 47), the distribution of temperature change material 80 can be suitably nonuniform through the cross-section. Nonetheless, despite this nonuniformity of temperature change material 80 in the temperature change composite 72, and thus the temperature change member 70, in the z-direction 49, the temperature change material 80 can optionally be substantially uniformly distributed in the temperature change composite 72 in the plane 47 for improved manufacturability.

The temperature change material 80 can also optionally define a particle size distribution within the temperature change composite 72. For example, the particle size distribution can be from a smaller temperature change material 80 particle size to a larger temperature change material 80 particle size measured from the member inner surface 87 to the member outer surface 88. As such, the temperature change member 80 can include relatively larger sized temperature change material 80 particles that have been found to dissolve slower when exposed to liquid, thereby providing a relatively longer lasting temperature change sensation to the wearer in use and/or making the temperature change member 70 more effective for multiple insults of urine. Nonetheless, the temperature change member 70 can also include relatively smaller sized temperature change material 80 that has been found to dissolve more quickly when exposed to liquid. As such, the temperature change member 70 can also provide more immediate temperature change feedback to the wearer upon urination. Moreover, depending on the location of the relatively smaller temperature change material 80, the temperature change member 70 can present a relatively smooth, pleasing surface toward the wearer as relatively smaller materials may be closer to the member inner surface 87.

Accordingly, at least 10 percent of the temperature change material 80 included in the temperature change member 70 can have a particle size of not greater than 200 microns. Moreover, at least 10 percent of the temperature change material 80 can have a particle size of greater than 500 microns. In another aspect, at least 25 percent of the temperature change material 80 included in the temperature change member 70 can have a particle size of not greater than 200 microns. Moreover, at least 25 percent of the temperature change material 80 can have a particle size of greater than 500 microns. Alternatively, at least 10 percent of the temperature change material 80 included in the temperature change member 70 can have a particle size of not greater than 90 microns. Moreover, at least 10 percent of the temperature change material 80 can have a particle size of greater than 710 microns. In another aspect, at least 25 percent of the temperature change material 80 included in the temperature change member 70 can have a particle size of not greater than 90 microns. Moreover, at least 25 percent of the temperature change material 80 can have a particle size of greater than 710 microns.

In a particular aspect, at least 50 percent of the temperature change material 80 by weight can define a particle size of at least 500 microns. That is, if the temperature change member 70 contained a total amount of temperature change material 80 of 20 grams, at least 10 grams of that temperature change material 80 will have a particle size of at least 500 microns. Alternatively, at least 75 percent of the temperature change material 80 by weight can define a particle size of at least 500 microns. In another alternative, between 50 percent and 85 percent of the temperature change material 80 can define a particle size of at least 500 microns by weight. In yet another alternative at least 50 percent of the temperature change material 80 by weight can define a particle size of between 300 and 710 microns. In still yet another alternative, between 50 percent and 85 percent of the temperature change material 80 by weight can define a particle size of between 300 and 710 microns. Accordingly, the temperature change member 70 can include suitably sized temperature change material 80 for more sustained temperature change performance. That is, as mentioned above, the relatively larger sized temperature change material 80 can provide a longer lasting temperature change sensation to the wearer upon being exposed to urine than the relatively smaller sized temperature change material 80.

A suitable method for determining the particle size of the temperature change material 80 and the particle size distribution of the temperature change material 80 in the temperature change member 70 is by sieve size analysis. A stack of sieves are used to determine the particle size distribution of a given sample. Thus, for example, in principle, a particle that is retained on a sieve with 710 micron openings is considered to have a particle size greater than 710 microns. A particle that passes through a sieve having 710 micron openings and is retained on a sieve having 500 micron openings is considered to have a particle size between 500 and 710 microns. Further, a particle that passes through a sieve having 500 micron openings is considered to have a particle size less than 500 microns.

The sieves are placed in order of the size of the openings with the largest openings on the top of the stack and the smallest openings on the bottom of the stack. Thus, all of the temperature change material 80 from a temperature change member can be weighed and placed into the sieve with the largest openings. Alternatively, if it is desired to determine the particle size or particle size distribution of the temperature change material 80 in only a particular portion of the temperature change member, only the temperature change material 80 from that portion can be weighed and placed into the sieve with the largest openings. U.S. Standard sieves can be used in the sieve stack, including 20 mesh (850 microns), 25 mesh (710 microns), 35 mesh (500 microns), 50 mesh (300 microns) and 170 mesh (90 microns).

The sieve stack is shook for 10 minutes with a Ro-Tap mechanical Sieve Shaker, Model RX29 available from W.S. Tyler of Mentor, Ohio, or other similar shaking device at standard test conditions. After shaking is complete, the temperature change material 80 retained on each sieve is removed and the weight is measured and recorded. The percentage of particles retained on each sieve is calculated by dividing the weights of the particles retained on each sieve by the initial sample weight.

Further, as mentioned above, the temperature change material 80 having various particle sizes may be suitably disposed in certain portions of the temperature change member 70. In a particular aspect, at least 70 percent of the temperature change material 80 by weight in the 25 percent of the temperature change member 70 extending in the z-direction 49 adjacent the member inner surface 87 can have a particle size of less than 300 microns. For example, if the temperature change member is 10 mm thick in the z-direction, then at least 70 percent of the temperature change material 80 in the 2.5 mm adjacent the inner surface 87 can have a particle size of less than 300 microns. Alternatively, the at least 70 percent of the temperature change material 80 by weight in the 25 percent of the temperature change member 70 extending in the z-direction 49 adjacent the member inner surface 87 can have a particle size of less than 200 microns, and in yet another alternative, less than 100 microns. In another aspect, at least 70 percent of the temperature change material 80 by weight in the 25 percent of the temperature change member 70 extending in the z-direction 49 adjacent the member inner surface 87 can have a particle size of between 200 microns and 500 microns. Temperature change material 80 featuring particle sizes proximate the member inner surface 87 as described above are suitably small enough to provide a more pleasing surface toward the wearer in use, as well as providing the relatively quick temperature change sensation to the wearer, as described above.

Similarly, relatively larger temperature change material 80 can be disposed in certain portions of the temperature change member 70, such as disposed proximate the member outer surface 88. Alternatively, the relatively large temperature change material 80 can be disposed toward a member z-directional centerline, indicated at the arrow marked 90. As such, at least 70 percent of the temperature change material 80 by weight in the 25 percent of the temperature change member 70 extending in the z-direction 49 adjacent the member outer surface 88 can have a particle size of less than 300 microns. Alternatively, at least 70 percent of the temperature change material 80 by weight in the 25 percent of the temperature change member 70 extending in the z-direction 49 adjacent the member outer surface 88 can have a particle size of less than 200 microns, and in yet another alternative, less than 100 microns. In another aspect, at least 70 percent of the temperature change material 80 by weight in the 25 percent of the temperature change member 70 extending in the z-direction 49 adjacent the member outer surface 88 can have a particle size of between 200 microns and 500 microns. Temperature change material 80 featuring particle sizes proximate the member inner surface 87 and outer surface 88 as described above are suitably small enough to more reliably provide a pleasing surface toward the wearer in use, such as in the event of flipping of the temperature change member 70 during the manufacture of the pants 20.

In another alternative, and to further provide an effective temperature change member 70 that yet provides a pleasing surface to the wearer, less than 10 percent of the total amount of temperature change material 80 in the temperature change member 80 by weight can be located in the 10 percent of the temperature change member thickness extending in the z-direction 49 adjacent the member inner surface 87. Similarly, less than 10 percent of the total amount of temperature change material 80 in the temperature change member 80 by weight can be located in the 10 percent of the temperature change member thickness extending in the z-direction 49 adjacent the member outer surface 88. As such, in aspects where less than 10 percent of the temperature change material 80 by weight is adjacent the member surfaces 87 and 88, assembly of the pants 20 can be simplified as flipping of the member 70 during assembly of the pants 20 does not negatively impact the performance of the member 70, and the member 70 can still provide a pleasing surface to the wearer.

One suitable method for determining the distribution of the temperature change material 80 within the temperature change member 70 is by way of a photomicrograph, electron micrograph, or similar imaging technique. For example, a Scanning Electron Microscope (JSM-840 from J.E.O.L., Peabody, Mass.) can be used. Cross-sections can be taken in the z-direction 49 by cutting with a fresh straight-edge razor blade, taking care to avoid dragging temperature change material 80 from one area of the temperature change member 70 to another area of the member 70. Accordingly, a magnified image of the z-directional cross-section of the temperature change member 70 can be taken. From this image, dimensions of the temperature change member 70 can be determined, and the distribution of the temperature change material 80 in the z-direction can also be observed. Moreover, the size of the temperature change material 80 in various relative locations of the temperature change member 70 can also be determined on the computer screen using software (SEMICAPS Genie v. 1.0 desktop imaging system manufactured by SEMICAPS, Inc., Santa Clara, Calif.) in conjunction with the scanning electron micrograph.

Further, in particular aspects, at least 0.5 mm, alternatively at least 1 mm, and in yet another alternative at least 2 mm of the temperature change member 70 adjacent the member inner surface 87 can optionally be substantially free of temperature change material 80 to define a member first isolation zone 92. In still yet another alternative, between 0.5 mm to 2.0 mm of the temperature change member 70 adjacent the member inner surface 87 can optionally be substantially free of temperature change material 80 to define a member first isolation zone 92. Similarly, at least 0.5 mm, and alternatively at least 1 mm, and in yet another alternative at least 2 mm of the temperature change member 70 adjacent the member outer surface 88 can be free of temperature change material to define a member second isolation zone 94. In still yet another alternative, between 0.5 mm to 2.0 mm of the temperature change member 70 adjacent the member outer surface 88 can optionally be substantially free of temperature change material 80 to define a member second isolation zone 94. The first isolation zone 92 can be provided by a separate layer attached to the temperature change composite 72, such as the second carrier layer 76. Alternatively, the first isolation zone 92 can be provided by a portion of the temperature change composite 72 that is substantially free of temperature change material 80. Similarly, the second isolation zone 94 can be provided by a separate layer attached to the temperature change composite 72, such as the first carrier layer 74. Alternatively, the second isolation zone 94 can be provided by a portion of the temperature change composite 72 that is free of temperature change material 80.

As mentioned above, a suitable method for determining the distribution of the temperature change material 80 within the temperature change member 70 is by way of a photomicrograph, electron micrograph, or similar imaging technique. Accordingly, a magnified image of the z-directional cross-section of the temperature change member 70 can be taken. From the image, dimensions of isolation zones 92 and 94 may be measured.

Nonuniform distribution of the temperature change material 80 in the temperature change member 70 as described above can provide a number of benefits. For example, the temperature change member can remain effective at providing a temperature change sensation to the wearer in use while being less likely to cause irritation from a coarse surface due to temperature change material being located proximate the wearer.

As mentioned above, the temperature change composite 72 includes temperature change material 80 and optionally, can further include a matrix of fibers 78 where the temperature change material 80 is intermixed within the matrix of fibers 78. The matrix of fibers 78 may be substantially continuous or discrete and discontinuous. In addition, the matrix of fibers 78 of the temperature change composite 72 may be provided by a variety of different fibers as are known in the art. For example, the matrix of fibers 78 can include adhesive fibers, absorbent fibers, binders (including binder fibers), polymer fibers, and the like or combinations thereof. As such, the temperature change material 80 may be suitably entrapped within the matrix 78 to limit material shake-out or loss during manufacture and/or wear of the pants 20. Suitable temperature change composites 72 are described in U.S. Pat. No. 5,681,298 and U.S. patent application Ser. No. 11/143,359, each of which has been previously incorporated herein.

In particular, in aspects where the matrix of fibers 78 includes adhesive fibers, the fibers may be provided by a hot-melt adhesive. Such an adhesive generally comprises one or more polymers to provide cohesive strength, a resin or analogous material, perhaps waxes, plasticizers or other materials to modify viscosity, and/or other additives including, but not limited to, antioxidants or other stabilizers. It is also contemplated that alternative adhesives may be used without departing from the scope of this invention.

The temperature change material 80 can be intermixed with the adhesive fibers providing the matrix of fibers 78 by being fed into and entrained in a stream of adhesive to form a blended mixture of adhesive fibers and temperature change material 80. In such an aspect, the matrix of fibers can be optionally applied to a substrate, such as the first carrier layer 74. In addition, the second carrier layer 76 can, but need not, overlay the temperature change composite 72 and be secured thereto by the adhesive in the matrix of fibers 78.

The temperature change material 80 can be intermixed with the adhesive in alternating, layered fashion as is known in the art in order to arrive at the various configurations described above. Moreover, temperature change material of different size can be intermixed in various locations by pre-separating the various sized temperature change material 80 (for example, by sieving the temperature change material 80) or purchasing the temperature change material at the desired particle size(s) and entraining the material 80 with the adhesive fibers to arrive at the various size distributions described above. The stream of temperature change material 80 may be pneumatically provided or gravity fed by way of agitation.

An example of a suitable adhesive for use in providing the matrix of fibers 78 are hot-melt adhesives available from H.B. Fuller Adhesives of Saint Paul, Minn. under the designation HL8151-XZP. In particular, this adhesive is a hydrophilic adhesive which promotes the rapid wettability of the temperature change member 70 resulting in faster temperature change. Alternatively, it is contemplated that the adhesive can be a hydrophobic adhesive without departing from the scope of the present invention.

Alternatively, the matrix of fibers 78 of the temperature change composite 72 can include absorbent fibers. In such an aspect, the matrix of fibers 78 may be provided by absorbent fibers by forming the matrix on a forming surface of a conventional air-forming device. Suitable absorbent fibers can include natural absorbent fibers such as cellulosic fibers (i.e., wood pulp fibers) or cotton fibers, synthetic absorbent fibers such as rayon or cellulose acetate or combinations thereof. In particular, the absorbent fibers can be a mixed bleached southern softwood and hardwood Kraft pulp designated as CR1654 available from Bowater Inc. of Greenville, S.C. U.S.A. Other suitable absorbent fibers can include NB 416, a bleached southern softwood Kraft pulp available from Weyerhaeuser Co. of Federal Way, Wash. U.S.A.; CR 1654, a bleached southern softwood Kraft pulp available from Bowater, Incorporated, of Greenville, S.C. U.S.A.; SULPHATATE HJ, a chemically modified hardwood pulp available from Rayonier Inc. of Jesup, Ga. U.S.A. and NF 405, a chemically treated bleached southern softwood Kraft pulp available from Weyerhaeuser Co.

Optionally, in such an aspect, the matrix of fibers 78 can further include binder material. For example, the binder material can suitably be a thermoplastic binder material. Such binder materials can soften when exposed to heat and can substantially returns to their original condition when cooled to room temperature. Such thermoplastic binder materials, when in the softened state, constrain or entrap the fibers and other materials proximate the binder to stabilize the temperature change composite 72. Binder materials can be provided in powder or fiber form. Examples of suitable binder materials for use with the present invention can be those having low melting temperatures such as polyethylene glycol (PEG) or paraffin wax, both of which are available from Alrich of Saint Louis, Mo.

The temperature change material 80 can be intermixed with the absorbent fibers in alternating, layered fashion as is known in the art in order to arrive at the various configurations described above. Moreover, temperature change material of different size can be intermixed in various locations by pre-separating the various sized temperature change material 80 (for example, by sieving the temperature change material 80) or purchasing the temperature change material at the desired particle size(s) and entraining the material 80 with the adhesive fibers to arrive at the various size distributions described above. The stream of temperature change material 80 may be pneumatically provided or gravity fed by way of agitation.

In yet another alternative the matrix of fibers 78 may be provided by a coform composite including polymer fibers and absorbent fibers. Coform materials and coforming processes are known in the art and by way of example are described in U.S. Pat. Nos. 4,100,324 to Anderson, et al.; 5,284,703 to Everhart, et al.; and 5,350,624 to Georger, et al.; each of which are hereby incorporated by reference to the extent that they are consistent (i.e., not in conflict) herewith.

In a particular aspect, the matrix of fibers 78 may be provided by a coform composite that can be a blend of meltblown polymer fibers and cellulosic fibers. Various suitable materials may be used to provide the meltblown fibers such as a polyolefin material. Alternatively, the polymer fibers may be stretch polymer fibers, such as those provided by a copolymer resin. For instance, Vistamaxx® elastic olefin copolymer resin available from ExxonMobil Corporation of Houston, Tex. or KRATON G-2755 available from Kraton Polymers of Houston, Tex. may be used to provide stretchable polymer fibers for the matrix of fibers 78. Other suitable polymeric materials or combinations thereof may alternatively be utilized as are known in the art.

Further, various absorbent fibers may be utilized, such as NF 405, a chemically treated bleached southern softwood Kraft pulp available from Weyerhaeuser Co. of Federal Way, Wash. U.S.A.; NB 416, a bleached southern softwood Kraft pulp available from Weyerhaeuser Co.; CR-0056, a fully debonded softwood pulp available from Bowater Inc. of Greenville S.C., Golden Isles 4822 debonded softwood pulp available from Koch Cellulose of Brunswick, Ga., U.S.A.; and SULPHATATE HJ, a chemically modified hardwood pulp available from Rayonier Inc. of Jesup, Ga. U.S.A.

The polymer fibers and the absorbent fibers may be coformed to provide the matrix of fibers 78 by providing a stream of absorbent fibers and a stream of extruded molten polymeric fibers. Further, to provide the temperature change composite 72, a stream of temperature change material 80 can also be provided. These streams can be merged into a single stream and collected on a forming surface such as a forming belt or forming drum to form the temperature change composite 72 of the temperature change member 70. Optionally, a forming layer, such as first carrier layer 74, can be placed on the forming surface and used to collect the materials included in the temperature change composite 72.

The stream of absorbent fibers may be provided by feeding a pulp sheet into a fiberizer, hammermill, or similar device as are known in the art. Suitable fiberizers are available from Hollingsworth of Greenville, S.C. and are described in U.S. Pat. No. 4,375,448 issued Mar. 1, 1983 to Appel et al. The stream of polymer fibers may be provided by meltblowing a copolymer resin or other polymer. In particular, the melt temperature for a copolymer resin such as Vistamaxx® can be from 450 degrees F. (232 degrees C.) to 540 degrees F. (282 degrees C.) to improve the entrainment of the temperature change material in the matrix. As mentioned above, suitable techniques for producing nonwoven fibrous webs, which include meltblown fibers, are described in the previously incorporated U.S. Pat. Nos. 4,100,324 and 5,350,624. The meltblowing techniques can be readily adjusted in accordance with conventional know-how to provide turbulent flows that can operatively intermix the fibers and the temperature change material 80. For example, the primary air pressure may be set at 5 psi and the meltblown nozzles may be 0.020 inch spinneret hole nozzles. The techniques can also be readily adjusted in accordance with conventional knowledge to provide the desired weight percentages of the various materials in the temperature change composite 72.

The stream of temperature change material 80 may be pneumatically provided or gravity fed by way of agitation. A suitable method and apparatus for delivering material in an air stream is described in U.S. Pat. No. 4,604,313 issued Aug. 5, 1986 to McFarland et al.; the disclosure of which is incorporated by reference herein to the extent that it is consistent (i.e., not in conflict) herewith. The coform material may also include other materials, such as superabsorbent materials.

The temperature change material 80 can be intermixed with the coform in alternating, layered fashion as is known in the art in order to arrive at the various configurations described above. Moreover, temperature change material of different size can be intermixed in various locations by pre-separating the various sized temperature change material 80 (for example, by sieving the temperature change material 80) or purchasing the temperature change material at the desired particle size(s) and entraining the material 80 with the adhesive fibers to arrive at the various size distributions described above. The stream of temperature change material 80 may be pneumatically provided or gravity fed by way of agitation.

In one aspect, the temperature change composite 72 provided by a coform composite as described above may be from 5 to 15 percent by weight meltblown polymer fibers, 10 to 50 percent by weight absorbent fibers and 40 to 80 percent by weight temperature change material. In a particular aspect, the temperature change composite 72 can be 8 percent by weight meltblown polymer fibers, 14 percent by weight absorbent fibers, 78 percent by weight temperature change material and define a basis weight of 1340 gsm.

As mentioned above, the temperature change member 70 can optionally include a first carrier layer 74 (FIGS. 4 and 5) in superposed relationship with the temperature change composite 72. Further, the temperature change member 70 can optionally include a first carrier layer 74 and a second carrier layer 76 (FIG. 5) where the first carrier layer and the second carrier layer 76 sandwich the temperature change composite 72. The first and second carrier layers 74 and 76 may be provided by separate webs of material, or alternatively can be provided by a single web of material that is folded in half about the temperature change composite 72. Referring to FIG. 9, the liner 42 may also serve as the second carrier layer 76.

In certain aspects, the carrier layers 74 and 76 can be liquid permeable or liquid impermeable. For instance, one carrier layer, such as the first carrier layer 74 may be at least partially liquid impermeable, and optionally substantially or completely liquid impermeable and the other carrier layer, (i.e., the second carrier layer 76 can be at least partially liquid permeable and optionally substantially or completely liquid permeable. In such an aspect, the first carrier layer 74 can be disposed toward the outer surface 32 and the second carrier layer 76 can be disposed toward the inner surface 30. As such, liquid insults may pass through the second carrier layer 76 to activate the temperature change material, and the first carrier layer 74 can slow the flow of the liquid insult from leaving the temperature change member 70 thus maximizing the temperature change that can be felt by the wearer. Alternatively, the first carrier layer 74 can be liquid permeable, and in aspects with a second carrier layer 76, both carrier layers 74 and 76 may be liquid permeable. In yet another alternative, the carrier layers 74 and 76 can each include portions that are liquid permeable and liquid impermeable. Carrier layers 74 and 76 as described above can further improve the integrity of the temperature change member 70 thereby enhancing processability, and can also aid in retaining the temperature change material within the member 70.

Suitable liquid permeable materials for the carrier layers 74 and 76 include tissue layers, nonwoven layers, or combinations thereof. In particular, materials described as suitable for use as the bodyside liner 42 may also be suitable for a liquid permeable carrier layer 74 and 76. Accordingly, a liquid permeable carrier layer 74 and 76 can also be stretchable. Likewise, materials described as suitable for use as the outercover 40 may be suitable for use as a liquid impermeable carrier layer 74 and 76. Accordingly, a liquid impermeable carrier layer 74 and 76 can also be stretchable.

The temperature change material 80 of the various aspects of the present invention can include a substance that provides a temperature change when placed near the wearer and contacted with urine. The temperature change can be either an absorption or release of heat that is noticeable to the wearer. Absorption of heat by the temperature change material 80 will provide the wearer with a cool sensation, while a release of heat by the substance will provide the wearer with a warm sensation. Reference is made to U.S. Patent Application Publication 2004/0254549, published Dec. 16, 2004, in the name of Olson, et al., incorporated by reference herein, for additional information regarding the mechanism by which the temperature change sensation is accomplished. Suitably, the temperature change material 80 can be provided in particulate form for ease of processing in the described aspects.

The temperature change material 80 is responsive to contact with an aqueous solution such as urine to either absorb or release heat. The mechanism by which this is accomplished is dissolution of the substance in the aqueous solution, swelling of the substance in the aqueous solution, or reaction of the substance in the aqueous solution. For example, the temperature change material may include particles that have a substantial energy difference between a dissolved state and a crystalline state so that energy in the form of heat is absorbed or released to the environment upon contact with urine, or the temperature change material may release or absorb energy during swelling or reacting in an aqueous solution.

While a wide variety of substances may result in a temperature change when contacted with an aqueous solution, the selection of a particular temperature change material 80, the determination of the amount to be used and the location of the substance should be based in part on the desired temperature change. Specifically, the temperature change member 70 may suitably provide the training pants 10 with a temperature change (i.e., cooler or warmer) when wet of at least about 5 degrees C., more suitably about 10 degrees C., still more suitably about 15 degrees C. Alternatively, the temperature change member 70 can provide the pant 20 with a surface temperature change when wet of from 5 degrees C. to 15 degrees C. Surface temperature changes within this range are believed to be identifiable to some extent by children of toilet training age. More suitably the temperature change member 70 can provide the pant 20 with a surface temperature change when wet of from 5 degrees C. to 10 degrees C.

Thus, in a particular aspect, where the temperature change material is endothermic, a drop in the temperature of the product when insulted can be from about 37 degrees C. to about 25 degrees C., and further to about 22 degrees C. for improved effectiveness, particularly with a preoccupied wearer (i.e., a playing child). The temperature change can suitably last for at least 10 minutes, and more suitably for approximately 15 minutes.

By way of example, polyols such as Xylitol particles may be selected to provide a cooling sensation as Xylitol particles absorb heat when dissolved in an aqueous solution. Alternatively, other polyols such as Sorbitol or Erithritol may be advantageously selected to provide a cooling sensation. In yet another alternative, various combinations of the above temperature change materials may be utilized. Suitable polyols can be obtained from Roquette America, Inc., a company having offices in Keokuk, Iowa, under the trade name of XYLISORB (xylitol) or NEOSORB (Sorbitol). Such polyols can generally be obtained from the manufacturer in particular particle sizes, such as 90 microns, 300 microns, 500 microns, and the like for distribution in the temperature change member 70.

Other suitable temperature change materials that absorb heat during dissolution include salt hydrates, such as sodium acetate (H₂O), sodium carbonate (10H₂O), sodium sulfate (10H₂O), sodium thiosulfate (5H₂O), and sodium phosphate (10H₂O); anhydrous salts such as ammonium nitrate, potassium nitrate, ammonium chloride, potassium chloride, and sodium nitrate; organic compounds such as urea and the like or combinations thereof.

The temperature change material 80 may also include those substances that absorb or release heat during swelling. By way of illustration, one suitable temperature change material that releases heat during swelling is a lightly cross-linked partially neutralized polyacrylic acid. Other temperature change material 80 that releases heat during dissolution includes aluminum chloride, aluminum sulfate, potassium aluminum sulfate, and the like or combinations thereof.

The temperature change material 80 can also include ortho esters or ketals such as menthone ketals that result from reacting menthone with alcohols containing 1 to 8 carbons or polyols containing 2 to 8 carbons, and all structural and optical isomers thereof. Particular menthone ketals that may be suitable include menthone-glycerol ketal and menthone-propylene glycol ketal. Particular ketals are disclosed in U.S. Pat. No. 5,348,750 issued to Greenberg, and U.S. Pat. No. 5,266,592 issued to Grub et al.

The temperature change member 70 can optionally be subjected to additional processing for improved performance. For example, the temperature change member 70 can be passed through a nip defined by opposed rolls in order to compress and densify all of the temperature change member 70 or certain regions of the temperature change member 70. Alternatively, other densification methods may be utilized as are well known to those skilled in the art. As such, the temperature change member 70 can define a density of between 0.20 grams per cm³ to 0.55 grams per cm³, particularly a density of between 0.25 grams per cm³ to 0.45 grams per cm³ and still more particularly, a density of 0.35 grams per cm³ in at least a portion of the temperature change member 70. Densities within these ranges are believed to allow provide a flexible yet robust temperature change member 70 that retains the temperature change material 80 within the matrix of fibers 78 and that has desirable integrity. Moreover, such densities are not so high as to crush or otherwise impair the temperature change material 80 thereby reducing its efficacy.

The smoothness or coefficient of friction of the temperature change member 70 may be dependent upon the materials from which the temperature change member 70 is constructed. For instance, as mentioned previously, smaller particles of temperature change material 80 may result in a smoother temperature change member 70 than one having larger particles of temperature change material 80. In addition, embedding the temperature change material into a matrix of fibers, as described herein, can result in a composite that is relatively smooth.

The smoothness of temperature change member 70 may be also be dependent upon a mechanical treatment and/or the way in which the temperature change member is constructed. For example, as described herein, temperature change materials 80 may be arranged within the temperature change member 70 to positively affect the coefficient of friction. Further, a mechanical treatment such as calendering can be used to smooth and increase the density of temperature change member 70. For example, if the temperature change material 80 is embedded in a soft coform material as described herein, calendering may push larger particles of temperature change material 80 deeper into the coform material to create a smoother surface. Alternatively, if the temperature change member 70 is sandwiched between carrier layers 74 and 76, calendering may have the effect of rearranging particles of temperature change material 80, and creating a smoother surface at layers 74 and 76.

The calendering process may be carried out by applying pressure to a material with a pair of nip rolls (not shown). The nip rolls have a fixed gap so that the material can be flattened to a relatively or substantially uniform thickness. The fixed gap may be about 0.037 inches (0.94 mm), or whatever size is desired to apply enough pressure to the material to achieve a smoothing effect. Using the calendering process, a temperature change member 70 can be smoothed to achieve a lower coefficient of friction at one or both of the two surfaces contacted by the nip rolls.

Friction Test. The surface friction of the temperature change member 70 may be determined with the following test methodology. This test is particularly suited for sheet materials such as nonwovens, bath tissue, facial tissue, towels, handsheets, and thick composite materials such as diapers, bandages, or pads. The test method utilizes a Kawabata Evaluation System equipment, and will hereinafter be referred to as the KES Method.

The KES surface friction and surface roughness electronic unit (model number KES-SE, available by Kato Tech Co. Ltd., Japan) is an electronic instrument equipped with a calculation circuit to measure geometrical roughness, friction coefficient, and the mean deviation of the coefficient of friction. Generally, a specimen is placed on a sliding table at a specified speed. The moving direction and distance are detected by a potentiometer as the displacement output voltage. Surface friction is measured with a friction-force sensor at a predetermined weight. The friction-force sensor is connected to a friction-force transducer with a linear differential transformer. The measured values are closely related to the hand touch feeling or a material's surface. The sensor used to test samples described is a multiple-wire sensor of 10 wires. Each wire has an outer diameter of 0.5 mm, and the total area of the wires is about 1 cm².

The laboratory environment is at 23+/−2 C and 50+/−relative humidity. Rubber gloves are used when handling the specimens so as not to affect the moisture content of the specimens.

Prepare specimens to a dimension of ten (10) cm by ten (10) cm and condition in the laboratory environment for at least 24 hours. If it is not possible to prepare specimens of this size, such as when removing a temperature change member 70 from a diaper for testing, then it is acceptable to use a sample having smaller dimensions as long as the test equipment does not have to undergo significant modifications. Significant modifications are defined as a modification that would alter the test results to the degree that a comparison to previous test results is not meaningful. Extensions to increase the area of the test specimen may be added for handling and testing provided that the extension surfaces are not tested or considered when determining the coefficient of friction of the temperature change member. Specimen thickness may vary from material-type to material-type. Within a given material, the thickness for each specimen has relatively low variance.

Place a conditioned specimen on the designated specimen table on the KES device. Align the specimen with the specimen table left edge, parallel to the specimen table long edge, and approximately centered with the sensor-hanging shaft. Avoid touching the test area. Adjust the sensor hanging shaft height so the center of the frictional force sensor-hanging shaft is approximately aligned with the top of the specimen. Once aligned, tighten the fixing screw.

Apply initial test settings to the KES device. First, set the KES device to test at a speed of 1 mm/second. Second, set the KES friction static load to 25 grams. Third, zero the electronic unit. Fourth, seat the frictional force sensor onto the sensor-hanging shaft. Fifth, ensure that the digital readout displays +/−00.00 volts. Sixth, set test sensitivity to “high.”

As the test is performed, record the following: (1) mean deviation of coefficient of friction (hereinafter “MD”), (2) the static load in grams, (3) speed, and (4) sensitivity (high). If the material tested has been treated, after removing the frictional force sensor from the hanging shaft, gently wipe the portion of the sensor that contacts the specimen with the tip of an applicator.

To create a report using the data from the test, refer to Table 1 for factors to calculate the coefficient of friction at a static load of 25 grams when sensitivity is set to high.

TABLE 1

| Speed (mm/s) | Factors for MD |
|---|---|
| 1 | 0.02 |
| 2 | 0.04 |
| 5 | 0.01 |
| 10 | 0.2 |

Since it is likely that different devices for determining the coefficient of friction may give slightly different values, it is accordingly to be expressly understood that the mean deviation of coefficient of friction (MD) values expressed hereinafter and in the claims are to be construed as values derived from the KES Method.

Experimental Data. In accordance with the KES Method described above, the following types of temperature change members 70 were tested to determine MD. MD data is obtained by testing specimens of temperature change members 70 in both cross direction and machine direction. A geometric mean of MD data is calculated by taking a square root of the product MD values in cross direction and machine direction. The geometric mean MD is hereinafter referred to as the “GMD.”

The material tested was constructed of a homogeneous blend of about 75% sorbitol, about 9% polymer, and about 16% pulp formed onto a carrier layer of about 13.6 gsm untreated SMS in accordance with the process described in U.S. Pat. No. 5,681,298 and U.S. patent application Ser. No. 11/143,359 (previously incorporated). The surface tested, the member inner surface 87, was opposite the carrier layer located at member outer surface 88. The sorbitol was a granular material obtained from Roquette Corporation (Keokuk, Iowa). The polymer was VISTAMAXX VM2370 obtained from ExxonMobil Chemical Company (Houston, Tex.). The wood pulp was NF405 pulp obtained from Weyerhaeuser Company (Federal Way, Wash.).

The temperature change member 70 specimens used in the experiment had the following physical properties as shown below in Table 2:

TABLE 2

| | Thickness | Density | Basis |
|---|---|---|---|
| Specimen | (mm) | (g/cm\hat{ }3) | Weight (gsm) |
| Uncalendered | 2.27 | 0.24 | 540 |
| Calendered | 1.57 | 0.34 | 540 |

GMD data for each specimen was obtained and analyzed using the Student's t-test, an inferential statistical technique, see Tables 3 and 4 below. By comparing the t-Critical, two-tail value to the t-Stat value, it is shown that there is a significant difference in GMD between calendered temperature change members 70 and uncalendered temperature change members 70. Likewise, Tables 5 and 6 show that there is a significant difference in GMD value between calendered and un-calendered material and the liner material, as described above. Thus, even though some of the distributions representing the data may overlap, each of the materials has significantly different coefficients of friction when compared to one another.

TABLE 3
GMD for three materials.

| | Uncalendered | Calendered | Liner |
|---|---|---|---|
| Sample | Temperature Change | Temperature Change | (5 specimens |
| Number | Member | Member | tested) |
| 1 | 0.024489998 | 0.020175232 | 0.014199648 |
| 2 | 0.035190908 | 0.032597546 | 0.01722324 |
| 3 | 0.02946184 | 0.024996 | 0.018242807 |
| 4 | 0.029099828 | 0.020688161 | 0.012326394 |
| 5 | 0.02974895 | 0.025980762 | 0.014382976 |
| 6 | 0.02414705 | 0.01947922 | — |
| 7 | 0.022524653 | 0.021039962 | — |
| 8 | 0.024534058 | 0.018198901 | — |
| 9 | 0.028808332 | 0.0164 | — |
| 10 | 0.031759723 | 0.023778982 | — |
| Average: | 0.027976534 | 0.022333477 | 0.015275013 |

TABLE 4
Uncalendered temperature change member specimens
compared to uncalendered temperature change member specimens.

| | Uncalendered | Calendered |
|---|---|---|
| Average GMD | 0.027976534 | 0.022333477 |
| Variance | 1.57839E−05 | 2.19242E−05 |
| Observations | 10 | 10 |
| Pooled Variance | 1.88541E−05 | |
| Hypothesized Mean | 0 | |
| Difference | | |
| t Stat | 2.906006615 | |
| P(T <= t) two-tail | 0.009422336 | |
| t Critical two-tail | 2.100922037 | |

TABLE 5
Calendered temperature change member specimens compared to liner.

| | Calendered | Liner |
|---|---|---|
| Average GMD | 0.022333477 | 0.015275013 |
| Variance | 2.19242E−05 | 5.81247E−06 |
| Observations | 10 | 5 |
| Pooled Variance | 1.69668E−05 | |
| Hypothesized Mean | 0 | |
| Difference | | |
| t Stat | 3.128598715 | |
| P(T <= t) two-tail | 0.007994318 | |
| t Critical two-tail | 2.160368652 | |

TABLE 6
Uncalendered temperature change member specimens compared to liner.

| | Uncalendered | Liner |
|---|---|---|
| Average GMD | 0.027976534 | 0.015275013 |
| Variance | 1.57839E−05 | 5.81247E−06 |
| Observations | 10 | 5 |
| Pooled Variance | 1.27158E−05 | |
| Hypothesized Mean | 0 | |
| Difference | | |
| t Stat | 6.503142467 | |
| P(T <= t) two-tail | 1.99378E−05 | |
| t Critical two-tail | 2.160368652 | |

As seen in Table 4, the material exhibiting the highest GMD is the uncalendered temperature change member 70. Consumer studies have shown that a temperature change member 70 with a GMD of about 0.0280 is generally unacceptable for most wearers even when it is placed under a single layer of liner 42 without any other materials in between (e.g. surge). Thus, it is desired that an absorbent article of the present invention, which incorporates any of the embodiments of the temperature change member 70 described herein, have a GMD of less than about 0.0275. It is further desired that the temperature change member 70 have a GMD of less than about 0.0270. It is further desired that the temperature change member 70 have a GMD of less than about 0.0260. It is further desired that the temperature change member 70 have a GMD of less than about 0.0250. It is further desired that the temperature change member 70 have a GMD of less than about 0.0240.

Conversely, as observed in Tables 5 and 6, the material exhibiting the lowest coefficient of friction is the liner material. While the liner 42 GMD of about 0.0153 is not meant to limit the present invention, it is a number that may represent an acceptable GMD for any material that makes contact with the wearer's skin. If the GMD becomes too low, then the material may stick to the wearer's skin and feel uncomfortable. For instance, a GMD similar to that of polished glass would likely feel uncomfortable to the majority of wearers. As such, regardless of where the temperature change member 70 is placed with respect to the liner 42 or the absorbent body 44, it is desirable that the temperature change member 70 described herein, have a GMD of between about 0.0115 and 0.0275. It is further desired that the temperature change member 70 have a GMD of between about 0.0125 and 0.0275. It is further desired that the temperature change member 70 have a GMD of between about 0.0125 and 0.0260. It is further desired that the temperature change member 70 have a GMD of between about 0.0125 and 0.0250. It is further desired that the temperature change member 70 have a GMD of between about 0.0125 and 0.0240. It is further desired that the temperature change member 70 have a GMD of between about 0.0140 and 0.0240.

The temperature change member 70 is disposed within the training pants 20 so that, upon urination, liquid makes contact with the temperature change material 80. For example, the temperature change member 70 can be disposed with the absorbent body 44, for example intermediate the outercover 40 and liner 42. In particular, the temperature change member 70 may be attached to the absorbent body 44 and disposed toward the inner surface of the pants 20. Alternatively, the temperature change member 70 may be attached to the liner 42 adjacent the absorbent body 44. In still yet another alternative, the temperature change member 70 can be disposed within a gap between potions of the absorbent body 44 and attached, for example, to the outercover 40. Such an aspect is described in U.S. patent application Ser. No. 10/955,534 filed Sep. 29, 2004, in the name of Weber, et al., the disclosure of which is incorporated by reference to the extent that it is consistent (i.e., not in conflict) herewith.

As can be readily appreciated, the temperature change member 70 can be of various shapes and sizes. For example, the temperature change member 70 can be rectangular and can have a width in the lateral direction 48 of from 2.5 cm to 10 cm and a length in the longitudinal direction 46 of from 2.5 cm to 25 cm. In one aspect the temperature change member 70 can measure about 8 cm by about 10 cm. Alternatively, the temperature change member 70 can be oval in shape, circular, triangular, or the like. In yet another alternative, the temperature change member 70 can be generally be provided in strips that extend in the lateral 48 or longitudinal direction 46 and that can be separated by a gap. Further, it will be understood by those of skill in the art that the training pants 20 of the present invention could include more than one temperature change member 70.

A suitable procedure for determining the temperature change when wet of a product containing a temperature change material is described below in the temperature change test as follows. The test should be conducted in an environment having a stable temperature of 21 degrees C. to 22 degrees C. and a stable humidity of about 50 percent. The product to be tested is prepared by removing any elastic side panels and cutting all other elastics to permit the product to lay as flat as possible. The product is positioned in a Plexiglas cradle to simulate the configuration of the product in actual use. The center of the product is placed in the deepest portion of the cradle.

A liquid dispensing nozzle operatively connected to a liquid dispensing pump is positioned to dispense saline onto the inner surface of the product. The tip of the nozzle should be located 1 cm away from the inner surface and 10 cm forward of the center of the product, along the product's longitudinal axis. The pump is activated to dispense 90 ml of a stabilized isotonic 0.9 percent saline at a rate of 15 ml/sec. The saline is certified blood bank saline available from The Baxter Healthcare Corporation, Scientific Products Division, McGraw Park, Ill., and is at a temperature of 37° C.

The surface temperature of the product at the location of the temperature change member is measured using a standard thermometer or temperature sensing thermistors connected to a digital display or recording device. The surface temperature 30 seconds after the saline is dispensed is recorded as the test temperature. A reference temperature is obtained by performing this test on a portion of the product not including the temperature change material or on a similar product without the temperature change material. The surface temperature change when wet for the product is the difference between the test temperature and the reference temperature.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. An absorbent article defining a longitudinal direction, a lateral direction perpendicular to said longitudinal direction, and a z-direction perpendicular to a plane defined by said lateral direction and said longitudinal direction, said absorbent article comprising:
an outercover;
an absorbent body disposed on said outercover;
a liner disposed on the absorbent body; and
a temperature change member disposed between the liner and the outercover, the temperature change member defining a member inner surface and a member outer surface opposite said member inner surface, the temperature change member comprising temperature change material disposed in a nonuniform distribution by weight in the z-direction of the temperature change member, and having a GMD of less than about 0.0275 at the member inner surface as determined by the Friction Test described herein.

2. The absorbent article of claim 1 wherein said temperature change member further comprises a matrix of fibers and wherein a temperature change material is intermixed with said matrix of fibers.

3. The absorbent article of claim 2 wherein the matrix of fibers comprises adhesive fibers.

4. The absorbent article of claim 2 wherein the matrix of fibers comprises absorbent fibers.

5. The absorbent article of claim 2 wherein the matrix of fibers comprises polymer fibers.

6. The absorbent article of claim 2 wherein the matrix of fibers comprises a coform matrix of fibers.

7. The absorbent article of claim 6 wherein the temperature change member has a GMD of less than about 0.0270 when tested at the member inner surface.

8. The absorbent article of claim 1 wherein said temperature change member has a GMD of less than about 0.0270 when tested at the member inner surface.

9. The absorbent article of claim 1 wherein said temperature change member has a GMD of less than about 0.0260 when tested at the member inner surface.

10. The absorbent article of claim 1 wherein said temperature change member has a GMD of less than about 0.0250 when tested at the member inner surface.

11. The absorbent article of claim 1 wherein said temperature change member has a GMD of less than about 0.0240 when tested at the member inner surface.

12. An absorbent article comprising:
a liquid impermeable outercover;
an absorbent body disposed on said outercover; and
a temperature change member defining a member inner surface and a member outer surface opposite said member inner surface, the temperature change member comprising temperature change material wherein at least 50 percent of said temperature change material defines a particle size of at least 500 microns by weight, wherein the temperature change member has a GMD, as determined by the Friction Test described herein, of less than 0.0270 when tested at the member inner surface.

13. The absorbent article of claim 12 wherein the temperature change member further comprises a matrix of fibers and wherein said temperature change member is intermixed with said matrix of fibers.

14. The absorbent article of claim 12 wherein said temperature change member further comprises a matrix of fibers and wherein the matrix of fibers comprises a hot-melt adhesive.

15. An absorbent article defining a longitudinal direction, a lateral direction perpendicular to said longitudinal direction, and a z-direction perpendicular to a plane defined by said lateral direction and said longitudinal direction, said absorbent article comprising:
a liquid impermeable outercover having a garment facing surface and a body facing surface; and
a temperature change member disposed on the outercover body facing surface, the temperature change member defining a member inner surface and a member outer surface opposite the member inner surface, the temperature change member comprising a temperature change material disposed in a nonuniform distribution by weight in the z-direction of the temperature change member, the temperature change member having a GMD of less than about 0.0275 at the member inner surface, as determined by the Friction Test described herein.

16. The absorbent article of claim 14 further comprising a liner, wherein the liner is disposed on the member inner surface of the temperature change member in direct contact therewith.

17. The absorbent article of claim 14 further comprising a liner, and wherein the temperature change member further comprises a first carrier layer and a second carrier layer, wherein the second carrier layer consists of the liner and the member inner surface.

18. The absorbent article of claim 17 wherein the temperature change member has a GMD at the member inner surface of between about 0.0275 and 0.0115.

19. The absorbent article of claim 14 wherein greater than 50 percent of said temperature change material by weight has a particle size of between 300 microns and 710 microns.

20. The absorbent article of claim 19 further comprising a liner disposed directly on the member inner surface of the temperature change member.

21. The absorbent article of claim 19 wherein the liner has GMD of between about 0.0275 and 0.0115.
